(19) United States
(12) Patent Application Publication
(10) Pub. No.: US 2014/0256980 A1
(43) Pub. Date: Sep. 11, 2014

(54) PROCESS FOR MANUFACTURING HMB AND SALTS THEREOF

(71) Applicant: Yao-En Li, Buffalo Grove (IL)

(72) Inventor: Yao-En Li, Buffalo Grove (IL)

(73) Assignee: ABBOTT LABORATORIES, ABBOTT PARK, IL (US)

(21) Appl. No.: 14/238,802

(22) PCT Filed: Aug. 15, 2012
(86) PCT No.: PCT/US12/50893

§ 371 Date: May 22, 2014

Related U.S. Application Data
(60) Provisional application No. 61/523,531, filed on Aug. 15, 2011.
(60) Provisional application No. 61/526,729, filed on Aug. 24, 2011.
(60) Provisional application No. 61/555,423, filed on Nov. 3, 2011.

(51) Int. Cl. C07C 51/29

(52) U.S. Cl. CPC C07C 51/29
USPC 562/527; 422/198

(57) ABSTRACT

A continuous process and system for manufacturing beta-hydroxy-beta-methylbutyrate (HMB) and salts thereof is provided. The continuous process includes providing at least one oxidant and diacetone alcohol, and combining the at least one oxidant with the diacetone alcohol in a first flow reactor to produce a product stream comprising HMB or a salt thereof. Optionally, the process includes a second flow reactor for the acidification of a salt of beta-hydroxy-beta-methylbutyrate to produce beta-hydroxy-beta-methylbutyrate in free acid form.

### CROSS REFERENCE TO RELATED APPLICATIONS

This application claims priority to and the benefit of U.S. Provisional Patent Application No. 61/555,423, filed on Nov. 3, 2011, U.S. Provisional Patent Application No. 61/526,729, filed on Aug. 24, 2011, and U.S. Provisional Patent Application No. 61/523,531, filed on Aug. 15, 2011, the contents of which are incorporated by reference herein.

### TECHNICAL FIELD

The present disclosure relates to processes and systems for manufacturing beta-hydroxy-beta-methylbutyrate or salts thereof, and more particularly, a continuous process and system for manufacturing beta-hydroxy-beta-methylbutyrate or salts thereof, or both.

### BACKGROUND

Conventional industrial processes for producing beta-hydroxy-beta-methylbutyrate (HMB) are carried out in batch mode systems (i.e., a reaction is carried out in a first batch reactor, and when the reaction is complete, the final product is transferred to a second batch reactor to begin a new reaction). The conventional processes generally utilize sodium hypochlorite (NaClO) oxidation of diacetone alcohol (DIA) as the key synthetic reaction. In general, the batch processes for HMB production provide a very poor yield, which in turn limits the scale on which HMB can be produced.

### SUMMARY

Provided herein are continuous processes and systems for manufacturing beta-hydroxy-beta-methylbutyrate (HMB) or salts thereof, or both. The continuous processes and systems provide a very good product yield, reduce cycle time, and allow for the large scale production of HMB or salts thereof.

In a first embodiment, a continuous process for manufacturing beta-hydroxy-beta-methylbutyrate or a salt thereof is provided. The process includes providing at least one oxidant and diacetone alcohol at an equivalence ratio of the at least one oxidant to the diacetone alcohol within a range of 3:1 to 4:1. The at least one oxidant and the diacetone alcohol are combined in a flow reactor to form a product stream having a temperature of −10° C. to 40° C. The product stream comprises beta-hydroxy-beta-methylbutyrate or a salt thereof.

In a second embodiment, a continuous process for manufacturing calcium beta-hydroxy-beta-methylbutyrate is provided. The continuous process includes combining at least one oxidant with diacetone alcohol in a flow reactor to form a product stream having a temperature of −10° C. to 40° C. The equivalence ratio of the at least one oxidant to the diacetone alcohol is within a range of 3:1 to 4:1. The product stream comprises a salt of beta-hydroxy-beta-methylbutyrate. The product stream is combined with at least one acid to form a second product stream having a temperature of −5° C. to 5° C. The second product stream comprises beta-hydroxy-beta-methylbutyrate in free acid form. The second product stream is combined with at least one organic solvent to create an organic solvent phase. The beta-hydroxy-beta-methylbutyrate in free acid form is preferentially soluble in the organic solvent phase. A majority of the at least one organic solvent is removed from the organic solvent phase to produce a concentrated organic solvent-product phase comprising beta-hydroxy-beta-methylbutyrate in free acid form. The concentrated organic solvent-product phase comprising beta-hydroxy-beta-methylbutyrate is mixed with at least one source of calcium cations to form a third product stream comprising calcium beta-hydroxy-beta-methylbutyrate. The third product stream has a pH of at least 6. Calcium beta-hydroxy-beta-methylbutyrate is recovered from the third product stream.

In a third embodiment, a system for manufacturing beta-hydroxy-beta-methylbutyrate or a salt thereof is provided. The system includes a first pump in fluid communication with a source of at least one oxidant and a first heat exchanger, and a second pump in fluid communication with a source of diacetone alcohol and a second heat exchanger. In addition, the system includes a flow reactor in fluid communication with the first heat exchanger and the second heat exchanger. The at least one oxidant and the diacetone alcohol undergo an oxidation reaction in the flow reactor to produce a product stream comprising beta-hydroxy-beta-methylbutyrate or a salt thereof.

### BRIEF DESCRIPTION OF THE DRAWINGS

FIG. 1 illustrates a schematic of one embodiment of a continuous process for manufacturing beta-hydroxy-beta-methylbutyrate or a salt thereof.

FIG. 2 illustrates a schematic of one embodiment of a continuous process for manufacturing calcium beta-hydroxy-beta-methylbutyrate.

### DETAILED DESCRIPTION

Provided herein are continuous processes and systems for manufacturing beta-hydroxy-beta-methylbutyrate (HMB) or salts thereof, or both. The continuous processes and systems provide a very good yield, reduce cycle time, and allow for large scale production of HMB or salts thereof. Moreover, the continuous processes and systems for manufacturing HMB or salts thereof reduce energy consumption via increased cooling efficiency, reduce capital costs, and provide more efficient process control when compared to conventional processes for manufacturing HMB or salts thereof. The second embodiment is a sub-embodiment of the first embodiment and the third embodiment provides a system which can be useful in practicing certain processes according to the first and second embodiments.

In a first embodiment, a continuous process for manufacturing beta-hydroxy-beta-methylbutyrate or a salt thereof is provided. The continuous process comprises providing at least one oxidant and diacetone alcohol at an equivalence ratio of the at least one oxidant to the diacetone alcohol within a range of 3:1 to 4:1; and combining the at least one oxidant and the diacetone alcohol in a flow reactor to form a product stream having a temperature of −10° C. to 40° C. The product stream comprises beta-hydroxy-beta-methylbutyrate or a salt thereof.

In a second embodiment, a continuous process for manufacturing calcium beta-hydroxy-beta-methylbutyrate is provided. The continuous process according to the second embodiment comprises combining at least one oxidant with diacetone alcohol in a flow reactor to form a product stream having a temperature of −10° C. to 40° C. The equivalence ratio of the at least one oxidant to the diacetone alcohol is within a range of 3:1 to 4:1, and the product stream comprises a salt of beta-hydroxy-beta-methylbutyrate. The product stream is combined with at least one acid to form a second product stream having a temperature of −5° C. to 5° C. The second product stream comprises beta-hydroxy-beta-methylbutyrate in free acid form. The second product stream is combined with at least one organic solvent to create an organic solvent phase. The beta-hydroxy-beta-methylbutyrate in free acid form is preferentially soluble in the organic solvent phase. A majority of the at least one organic solvent is removed from the organic solvent phase to produce a concentrated organic solvent-product phase comprising beta-hydroxy-beta-methylbutyrate in free acid form. The concentrated organic solvent-product phase comprising beta-hydroxy-beta-methylbutyrate is mixed with at least one source of calcium cations to form a third product stream comprising calcium beta-hydroxy-beta-methylbutyrate. The third product stream has a pH of at least 6. Calcium beta-hydroxy-beta-methylbutyrate is recovered from the third product stream.

In a third embodiment, a system for manufacturing beta-hydroxy-beta-methylbutyrate or a salt thereof is provided. The system includes a first pump in fluid communication with a source of at least one oxidant and a first heat exchanger, and a second pump in fluid communication with a source of diacetone alcohol and a second heat exchanger. In addition, the system includes a flow reactor in fluid communication with the first heat exchanger and the second heat exchanger. The at least one oxidant and the diacetone alcohol undergo an oxidation reaction in the flow reactor to produce a product stream comprising beta-hydroxy-beta-methylbutyrate or a salt thereof.

As discussed above with respect to the first, second, and third embodiments, at least one oxidant and diacetone alcohol are combined in a flow reactor to form a product stream comprising beta-hydroxy-beta-methylbutyrate or a salt thereof. The at least one oxidant and the diacetone alcohol undergo an oxidation reaction in the flow reactor. One example of such an oxidation reaction is illustrated in Scheme 1.

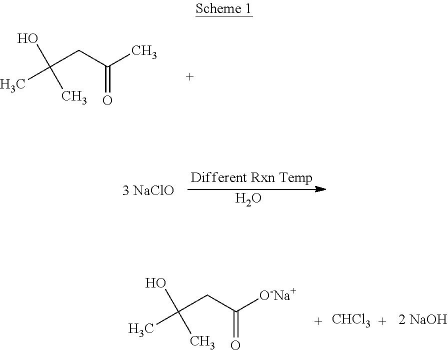

As can be seen in Scheme 1, in certain embodiments, the at least one oxidant is sodium hypochlorite, and the product of the oxidation reaction comprises sodium beta-hydroxy-beta-methylbutyrate. Although the example illustrated by Scheme 1 utilizes sodium hypochlorite as the at least one oxidant, various materials may be utilized as the at least one oxidant. For example, in certain embodiments according the first, second, and third embodiments, the at least one oxidant is selected from the group consisting of sodium hypochlorite, calcium hypochlorite, calcium hypobromite, calcium hypoiodite, sodium hypobromite, sodium hypoiodite, and combinations thereof. When a calcium-based oxidant is utilized in the oxidation reaction, the product of the oxidation reaction comprises calcium beta-hydroxy-beta-methylbutyrate.

In the first and second embodiments of the processes, the at least one oxidant and diacetone alcohol are provided at an equivalence ratio of 3:1 to 4:1. As used herein, the term “equivalence ratio” refers to the molar ratio of the at least one oxidant to diacetone alcohol. In certain embodiments according to the first and second embodiments of the processes, the at least one oxidant and the diacetone alcohol may be each provided neat, or alternatively dissolved or dispersed in a solvent. For example, in certain embodiments of the first and second embodiments of the processes, the at least one oxidant is provided as an aqueous solution and the diacetone alcohol is neat. As used herein, the term “neat” refers to a pure or undiluted chemical compound. In some embodiments, the at least one oxidant is an aqueous solution having a concentration (by weight) of oxidant between 5% to 100%, including between 5% to 50%, also including 8% to 35%, also including 10% to 16%, and further including 12% to 15%. In some embodiments, the diacetone alcohol may have a concentration (by weight) from 80% to 100%, also including 95% to 100%, and further including 99% to 100%.

The oxidation of the diacetone alcohol by the at least one oxidant is an exothermic reaction that influences the product yield of beta-hydroxy-beta-methylbutyrate or a salt therof. A higher reaction temperature degrades the product and produces unwanted byproducts, which may include acetic acid or diols. Accordingly, in the first and second embodiments of the processes, the oxidation reaction is carried out at a controlled temperature. For example, in the first and second embodiments of the processes, the temperature of the product stream is within a range of −10° C. to 40° C. In certain embodiments according to the first and second embodiments, the temperature of the product stream is within a range of −10° C. to 0° C. In yet other embodiments according to the first and second embodiments, the temperature of the product stream is around −15° C. By controlling the temperature of the product stream within the stated ranges, it has been found that a higher product yield of beta-hydroxy-beta-methylbutyrate or a salt therof, when compared to conventional processes, is achievable. As discussed in more detail below, in certain embodiments according to the first and second embodiments, the temperature of the product stream is controlled by reducing the temperature of the flow reactor, such as by jacketing or otherwise cooling the flow reactor.

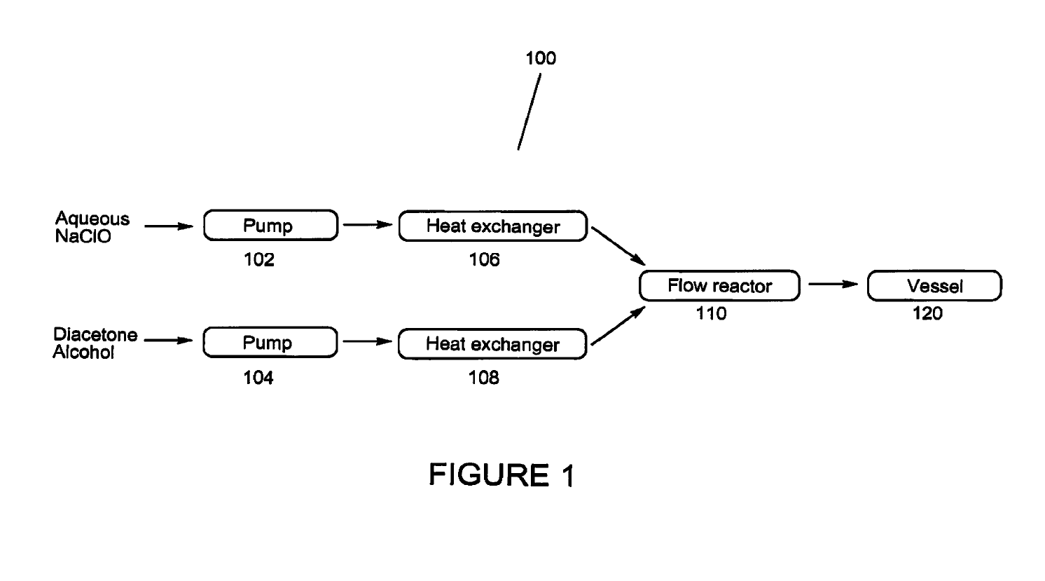

In order to provide optimal temperature control of the oxidation reaction, in certain embodiments of the first and second embodiments, prior to or upon combining in the flow reactor, the at least one oxidant is at a temperature of −20° C. to 20° C., and the diacetone alcohol is at a temperature of −20° C. to 20° C. In certain other embodiments according to the first and second embodiments, prior to or upon combining in the flow reactor, the at least one oxidant is at a temperature of −20° C. to 0° C., and the diacetone alcohol is at a temperature of −20° C. to 0° C. In order to achieve such temperature, in certain embodiments, the at least one oxidant and the diacetone alcohol are cooled to a temperature of −20° C. to 20° C. prior to or upon being combined in the flow reactor. The cooling of the at least one oxidant and the diacetone may be performed utilizing virtually any type of cooling process sufficient to achieve the specified temperatures. For example, and as shown in FIG. 1, in certain embodiments according to the first and second embodiments, the at least one oxidant and the diacetone alcohol may each flow through one or more heat exchangers, such as a chiller, to achieve a temperature of −20° C. to 20° C.

In certain embodiments according to the first and second embodiments, the at least one oxidant and diacetone alcohol remain in the flow reactor for 3 minutes to 20 minutes to carry out the oxidation reaction. In other words, the residence time of the oxidation reaction within the flow reactor is 3 minutes to 20 minutes. As used herein, the term “residence time” refers to the volume of the flow reactor divided by the volumetric flow rate (i.e., volumetric flow rate of the at least one oxidant plus the volumetric flow rate of diacetone alcohol) entering the flow reactor. In other embodiments, the at least one oxidant and diacetone alcohol remain in the flow reactor for 4 minutes to 18 minutes, also including 8 minutes to 14 minutes, and further including 10 minutes to 12 minutes.

In certain embodiments according to the first embodiment of the continuous process for manufacturing beta-hydroxy-beta-methylbutyrate or a salt thereof, the process further comprises the step of collecting the product stream, which comprises a salt of beta-hydroxy-beta-methylbutyrate. For example, in certain embodiments, and as seen in FIG. 1, the product stream exiting the flow reactor may be collected in a vessel (120), such as a holding tank or a batch reactor that may be used to further process the collected product stream comprising a salt of beta-hydroxy-beta-methylbutyrate.

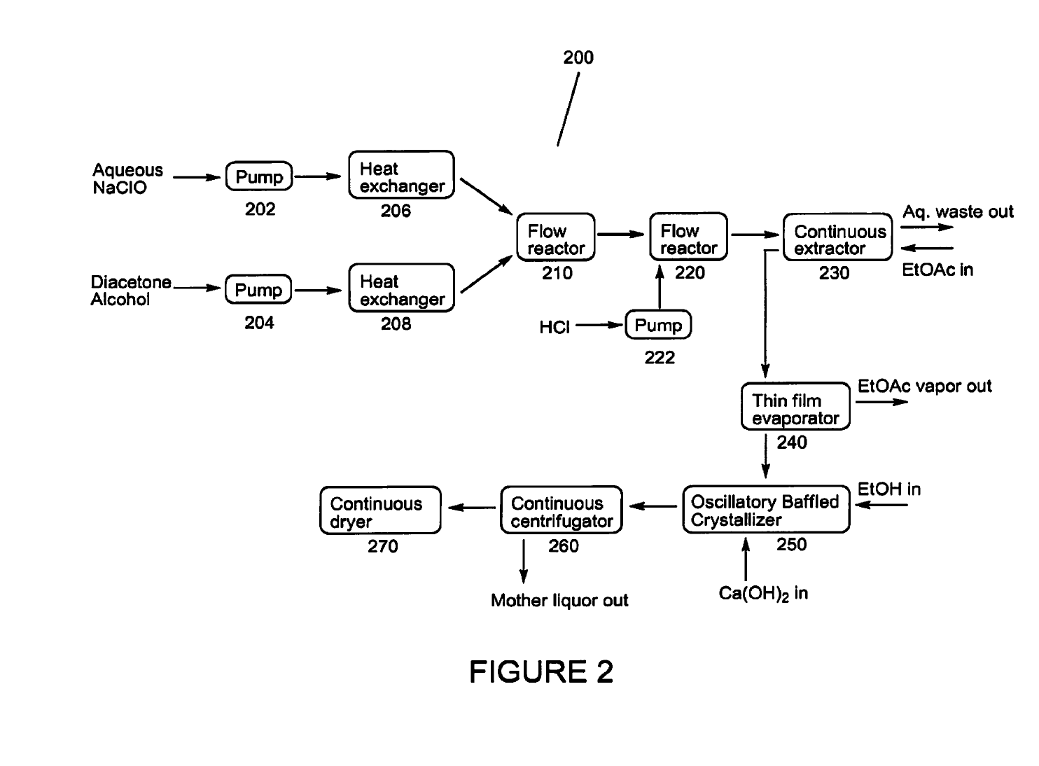

Referring now to FIG. 2, in certain embodiments according to the first and second embodiment, the continuous process may further comprise the step of combining the product stream with at least one acid to form a second product stream having a temperature of −5° C. to 5° C. and a pH of less than 5. The second product stream comprises beta-hydroxy-beta-methylbutyrate in free acid form. In other words, the product stream comprising a salt of beta-hydroxy-beta-methylbutyrate undergoes an acidification reaction at a temperature of −5° C. to 5° C. and a pH of less than 5 to produce a second product stream comprising beta-hydroxy-beta-methylbutyrate in free acid form. In other embodiments according to the first and second embodiments, the acidification reaction is carried out at a temperature of −5° C. to 0° C. and a pH of less than 3. As seen in FIG. 2, in certain embodiments, the product stream exiting the flow reactor may be combined with at least one acid in a second flow reactor. Alternatively, in other embodiments, a single flow reactor may be used and the at least one acid may be introduced into the single flow reactor at a predetermined downstream location to combine with the product stream. In addition, in yet other embodiments, the at least one acid may be combined with the product stream collected in a vessel (120), as previously described with reference to FIG. 1, to carry out the acidification reaction to form beta-hydroxy-beta-methylbutyrate in free acid form.

Various types of acids may be utilized for the at least one acid. In certain embodiments of the first and second embodiments, the at least one acid may be an aqueous acid solution, a gas, or neat. For example, in certain embodiments according to the first and second embodiments, the at least one acid is selected from the group consisting of hydrogen chloride gas, hydrochloric acid, hydrobromic acid, hydroiodic acid, sulfuric acid, bromic acid, and combinations thereof. In certain embodiments according to the first and second embodiments, the at least one acid combined with the product stream is a gaseous acid. For example, the gaseous acid may be hydrogen chloride gas. Use of a gaseous acid, as opposed to an aqueous acid solution, minimizes aqueous waste, as well as minimizes the amount of solvent required in subsequent steps of the process.

In certain embodiments according to the first and second embodiments of the continuous process, one or more reaction solvents may be used in connection with any of the various reactions carried out in the process. The total amount of the reaction solvent utilized (when reaction solvent is utilized) can be appropriately set under consideration of reactivity and operability and is generally set within a wide range from 1 to 1000 parts by weight, from 5 to 500 parts by weight, from 5 to 50 parts by weight, and from 10 to 20 parts by weight, per 1 part by weight of the substrate. In certain embodiments according to the first and second embodiments, the reaction solvent is selected from the group consisting of water, ethanol, ethyl acetate, and combinations thereof. For example, in certain embodiments of the first and second embodiments, water is used as a reaction solvent in the oxidation reaction (with the at least one oxidant as a substrate and the diacetone alcohol as a substrate) and the acidification reaction (with a salt of beta-hydroxy-beta-methylbutyrate as a substrate and hydrogen chloride as a substrate) disclosed herein. In addition, in certain embodiments according to the first and second embodiments of the continuous process water is used as a reaction solvent in a neutralization reaction (with beta-hydroxy-beta-methylbutyrate in free acid form as a substrate and at least one source of calcium cations as a substrate) and a crystallization process (with a salt of beta-hydroxy-beta-methylbutyrate as a substrate), as described below. Moreover, in certain embodiments according to the first and second embodiments, water, ethanol, and ethyl acetate are used as reaction solvents in the neutralization reaction. Furthermore, in other certain embodiments according to the first and second embodiments, water and ethanol are used as reaction solvents in the crystallization process.

Referring now to Scheme 2 (below), one embodiment of a synthetic process for preparing calcium beta-hydroxy-beta-methylbutyrate is shown. In other embodiments, a similar process may be followed, but other salts of beta-hydroxy-beta-methylbutyrate may be prepared including, but not limited to, alkali metal salts, alkaline earth metal salts, or both. The first two reactions seen in Scheme 2, are the oxidation of diacetone alcohol (1) with at least one oxidant (here sodium hypochlorite (2)) to produce a salt of beta-hydroxy-beta-methylbutyrate (here the sodium salt (3)), and the acidification of the salt of beta-hydroxy-beta-methylbutyrate with at least one acid (here hydrochloric acid) to produce beta-hydroxy-beta-methylbutyrate in free acid form (4). Scheme 2 further illustrates a neutralization step, or salt formation step, carried out by treating the beta-hydroxy-beta-methylbutyrate in free acid form (4) with at least one source of calcium cations (here calcium hydroxide) to form the calcium salt of beta-hydroxy-beta-methylbutyrate (5). Finally, Scheme 2 illustrates an optional step of recrystallizing the calcium salt of beta-hydroxy-beta-methylbutyrate with, for example, a recrystallization solvent, such as ethanol, to provide crystalline calcium beta-hydroxy-beta-methylbutyrate (6).

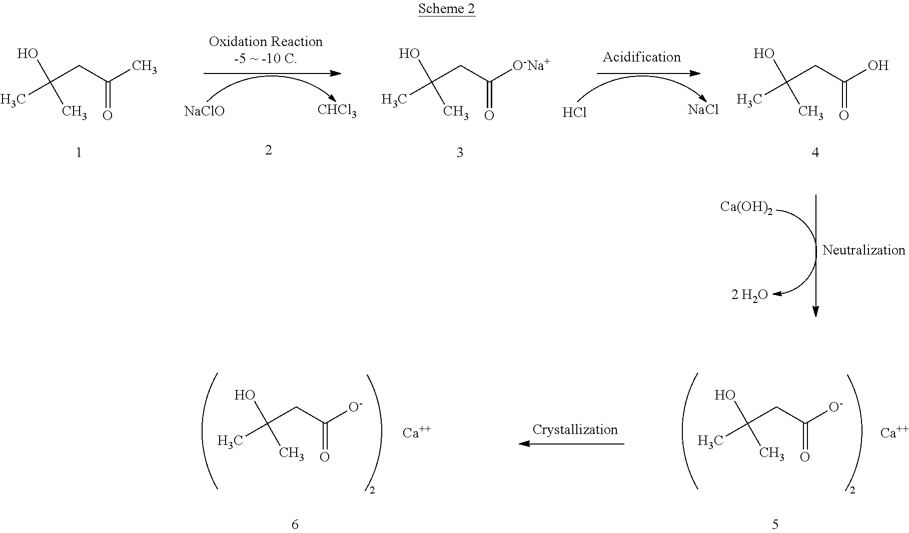

As previously mentioned, in the continuous process according to the second embodiment, and in certain embodiments of the continuous process according to the first embodiment comprise combining at least one oxidant with diacetone alcohol in a flow reactor to form a product stream and subsequently combining the product stream with at least one acid to form a second product stream comprising beta-hydroxy-beta-methylbutyrate in free acid form. Further, according to the continuous process of the second embodiment, and according to certain embodiments of the first embodiment, the process comprises combining the second product stream with at least one organic solvent to create an organic solvent phase. The beta-hydroxy-beta-methylbutyrate in free acid form is preferentially soluble in the at least one organic solvent such that the beta-hydroxy-beta-methylbutyrate in free acid form enters the organic solvent phase.

In certain embodiments according the first and second embodiments, the second product stream and the at least one organic solvent may be combined in a continuous countercurrent extractor such that the beta-hydroxy-beta-methylbutyrate in free acid form enters the organic solvent phase. As mentioned above, the beta-hydroxy-beta-methylbutyrate in free acid form is preferentially soluble in the at least one organic solvent. In certain embodiments according the first and second embodiments, the at least one organic solvent is selected from the group consisting of ethyl acetate, diethyl ether, and combinations thereof. One or more other organic solvents may be utilized for the at least one organic solvent as long as the free acid form of the beta-hydroxy-beta-methylbutyrate is preferentially soluble in such solvent(s).

In a further step of the continuous process of the second embodiment, and in certain embodiments according to the first embodiment, a majority of the at least one organic solvent is removed from the organic solvent phase to produce a concentrated organic solvent-product phase comprising beta-hydroxy-beta-methylbutyrate in free acid form. Removal of a majority of the at least one organic solvent from the organic solvent phase may be accomplished by a variety of techniques. For example, in certain embodiments according to the first and second embodiments, a majority of the at least one organic solvent is removed from the organic solvent phase in an evaporator, such as a thin film or wiped film evaporator. In alternative embodiments, a majority of the at least one organic solvent is removed from the organic solvent phase via distillation. After a majority of the at least one organic solvent is removed from the organic solvent phase, the concentrated organic solvent-product phase comprising beta-hydroxy-beta-methylbutyrate in free acid form may undergo further processing and the removed organic solvent may be recovered or recycled to the process.

According to the continuous process of the second embodiment, and according to certain embodiments of the first embodiment, the concentrated organic solvent-product phase comprising beta-hydroxy-beta-methylbutyrate is mixed with at least one source of calcium cations to form a third product stream comprising calcium beta-hydroxy-beta-methylbutyrate. As previously noted with respect to Scheme 2, this mixing entails a neutralization, or salt formation, producing the calcium salt of beta-hydroxy-beta-methylbutyrate. Preferably, the mixing is carried out at a pH of at least 6 such that the third product stream comprising calcium beta-hydroxy-beta-methylbutyrate has a pH of at least 6. In certain embodiments, the neutralization, or salt formation, is carried out at a pH of at least 7 so that the third product stream has a pH of at least 7.

In certain embodiments according to the first and second embodiments of the continuous process, the at least one source of calcium cations comprises a calcium-based base, and optionally comprises water as a solvent. In other embodiments according to the first and second embodiments, the at least one source of calcium cations includes at least one calcium salt and at least one base, and optionally comprises water as a solvent. In certain embodiments according to the first and second embodiments, the at least one source of calcium cations is selected from the group consisting of calcium hydroxide, calcium oxide, calcium carbonate, calcium acetate, and combinations thereof.

In certain embodiments according to the first and second embodiments of the continuous process, the mixing of the concentrated organic solvent-product phase comprising beta-hydroxy-beta-methylbutyrate with at least one source of calcium cations to form a third product stream comprising calcium beta-hydroxy-beta-methylbutyrate (or other salt form of beta-hydroxy-beta-methylbutyrate) further includes simultaneously providing a recrystallization solvent for mixing with the concentrated organic solvent-product phase and the at least one source of calcium cations. In certain embodiments according to the first and second embodiments of the continuous process, the recrystallization solvent is selected from the group consisting of ethanol, ethyl acetate, acetone, water, and combinations thereof. Thus, in this particular embodiment, the neutralization, or salt formation, is combined with recrystallization to produce a solution comprising crystalline calcium beta-hydroxy-beta-methylbutyrate (or other salt form of beta-hydroxy-beta-methylbutyrate). In certain embodiments according to the first and second embodiments, to achieve the combined neutralization-recrystallization process, the concentrated organic solvent-product phase comprising beta-hydroxy-beta-methylbutyrate, the at least one source of calcium cations, and the recrystallization solvent are fed to a continuous oscillatory baffled crystallizer, such as described by Lawton et al. “Continuous Crystallization of Pharmaceuticals Using a Continuous Oscillatory Baffled Crystallizer,” Organic Process Research & Development, 2009, 13(6), pp 1357-1363, which is incorporated herein by reference in its entirety, to produce a solution comprising crystalline calcium beta-hydroxy-beta-methylbutyrate (or other salt form of beta-hydroxy-beta-methylbutyrate).

According to the continuous process of the second embodiment, and according to certain embodiments of the first embodiment, comprises recovering the calcium beta-hydroxy-beta-methylbutyrate (or other salt-form of the beta-hydroxy-beta-methylbutyrate) from the third product stream. Recovering the calcium beta-hydroxy-beta-methylbutyrate (or other salt-form of the beta-hydroxy-beta-methylbutyrate) may be carried out utilizing various techniques. For example, in certain embodiments, the calcium beta-hydroxy-beta-methylbutyrate (or other salt-form of the beta-hydroxy-beta-methylbutyrate) is recovered from the third product stream by continuous centrifugation. In the continuous centrifugation, the calcium beta-hydroxy-beta-methylbutyrate (or other salt-form of the beta-hydroxy-beta-methylbutyrate) is separated from the solution (i.e., mother liquor), which solution may be further processed to recover any residual calcium beta-hydroxy-beta-methylbutyrate (or other salt-form of the beta-hydroxy-beta-methylbutyrate). In addition, in certain other embodiments, the calcium beta-hydroxy-beta-methylbutyrate (or other salt-form of the beta-hydroxy-beta-methylbutyrate) is recovered from the third product stream by filtration or decantation. Moreover, in certain embodiments, the calcium beta-hydroxy-beta-methylbutyrate (or other salt-form of the beta-hydroxy-beta-methylbutyrate) is recovered from the third product stream by employing a spray drying operation.

In certain embodiments of the continuous process according to the first and second embodiments, the process further comprises removing residual solvent from the recovered calcium beta-hydroxy-beta-methylbutyrate (or other salt-form of the beta-hydroxy-beta-methylbutyrate). The step of removing residual solvent from the recovered calcium beta-hydroxy-beta-methylbutyrate (or other salt-form of the beta-hydroxy-beta-methylbutyrate) may be performed by various methods. For example, in certain embodiments, the step of removing residual solvent from the recovered calcium beta-hydroxy-beta-methylbutyrate (or other salt-form of the beta-hydroxy-beta-methylbutyrate) comprises drying the recovered calcium beta-hydroxy-beta-methylbutyrate (or other salt-form of the beta-hydroxy-beta-methylbutyrate), such as by feeding the recovered calcium beta-hydroxy-beta-methylbutyrate (or other salt-form of the beta-hydroxy-beta-methylbutyrate) to a continuous dryer. It may not be possible to completely remove all residual solvent, thus the solid calcium beta-hydroxy-beta-methylbutyrate (or solid form of another salt-form of the beta-hydroxy-beta-methylbutyrate) may contain some amount of residual solvent.

Referring now to FIG. 1, a certain embodiment according to the third embodiment of a system for manufacturing beta-hydroxy-beta-methylbutyrate or a salt thereof is illustrated. (The third embodiment is not limited to the specific embodiment illustrated in FIG. 1.) As can be seen in FIG. 1, the system comprises a first pump (102) in fluid communication with a source of at least one oxidant (here aqueous sodium hypochlorite), and a first heat exchanger (106). Also, as can be seen in FIG. 1, the system includes a second pump (104) in fluid communication with a source of diacetone alcohol, and a second heat exchanger (108). As previously mentioned, the first and second heat exchangers (106, 108) are used to reduce the temperature of the at least one oxidant and diacetone alcohol.

With continued reference to FIG. 1, the illustrated and exemplary system according to the third embodiment also includes a flow reactor (110) in fluid communication with the first heat exchanger (106) and the second heat exchanger (108). As previously described herein, the at least one oxidant and the diacetone alcohol are combined and undergo an oxidation reaction in the flow reactor (110) to produce a product stream comprising beta-hydroxy-beta-methylbutyrate or a salt thereof.

In certain embodiments according to the first, second and third embodiments of the disclosure, the flow reactor comprises a tubular reactor having one or more static mixing elements. Moreover, in certain other embodiments according to the first, second, and third embodiments, the flow reactor includes a means of temperature control, such as an external or internal cooling jacket or a cooling tank (refrigerant tank). By controlling the reaction temperature (i.e., the temperature of the product stream) within the previously discussed ranges, thermal degradation of the beta-hydroxy-beta-methylbutyrate or a salt thereof may be reduced or even eliminated, thus increasing product yield. Suitable tubular reactors are commercially available from, for example, Koflo Corporation, 309 Cary Point Drive, Cary, Ill. 60013. In certain other embodiments, the flow reactor may comprise a single conduit or a plurality of conduits through which the process streams flow in parallel. According to certain embodiments of the first, second and third embodiments of the disclosure, the continuous production of beta-hydroxy-beta-methylbutyrate or a salt thereof may be adjusted via a plurality of flow reactors operating in parallel.

A wide variety of materials may be used for the flow reactor. For example, the material for the flow reactor includes, but is not limited to, a stainless steel tube or a tube lined with glass or TEFLON. In certain embodiments according to the first, second, and third embodiments disclosed herein, the flow reactor is a tubular reactor having an inner diameter of 0.2 millimeters to 50 millimeters, also including 5 millimeters to 25 millimeters, and further including 5 millimeters to 10 millimeters. Such an inner diameter provides sufficient area for satisfactory heat transfer to better control the reaction temperature of the oxidation reaction, acidification reaction, or both. With respect to the length of the flow reactor, it can be determined based upon the amount of time the at least one oxidant and diacetone alcohol remain in the flow reactor to carry out the oxidation reaction (i.e., the residence time required for the reaction).

In certain embodiments according to the first, second, and third embodiments, the flow reactor optionally includes an apparatus for accelerating the mixing of the at least one oxidant and diacetone alcohol (hereinafter referred to as “premixer”) in an inlet portion of the flow reactor. Examples of the premixer include, but are not limited to, stirred mixers, ultrasonic mixers, motionless mixers such as a static mixer, and piping joints.

A motionless mixer such as a static mixer can also be used as the flow reactor in certain embodiments according to the first, second, and third embodiments disclosed herein. Such a motionless mixer may provide better heat transfer characteristics, as well as a larger inner diameter. Commercially available motionless mixers include, but are not specifically limited to, a Sulzer static mixer and a Kenics static mixer. The motionless mixer may also have a premixer in an inlet portion thereof. The number of elements in the static mixer is not specifically limited but may be 10 or more, or 17 or more.

As previously mentioned with reference to FIG. 1, in certain embodiments according to the first, second, and third embodiments, the product stream exiting the flow reactor may be collected in a vessel (120). The vessel (120) may be, for example, one or more holding tanks or one or more batch reaction vessels used to further process the collected product stream comprising a salt of beta-hydroxy-beta-methylbutyrate. For example, after collecting a predetermined amount of the product stream in a first batch reaction vessel, the product stream may be diverted to a second batch reactor for collection. The predetermined amount of the product stream collected in the first batch reactor may then undergo an acidification reaction by feeding to the batch reactor an amount of at least one acid to produce a second product stream comprising beta-hydroxy-beta-methylbutyrate in free acid form.

With reference now to FIG. 2, a certain embodiment of a system according to the third embodiment is shown. As will be appreciated, several components of the illustrated system shown in FIG. 2 are similar to the components of the system shown in FIG. 1. For example, and as illustrated in FIG. 2, the exemplary system comprises a first pump (202) in fluid communication with a source of at least one oxidant (here aqueous sodium hypochlorite), and a first heat exchanger (206). Also seen in FIG. 2, the exemplary system includes a second pump (204) in fluid communication with a source of diacetone alcohol, and a second heat exchanger (208). The system according to the third embodiment the system according to the third embodiment also includes a flow reactor (210) in fluid communication with the first heat exchanger (206) and the second heat exchanger (208). As previously described herein, the at least one oxidant and the diacetone alcohol are combined and undergo an oxidation reaction at the specified conditions in the flow reactor (210) to produce a product stream comprising beta-hydroxy-beta-methylbutyrate or a salt thereof.

With continued reference to FIG. 2, certain embodiments of the system according to the third embodiment comprise a third pump in fluid communication with a source of at least one acid and the flow reactor. As previously described, the product stream comprising beta-hydroxy-beta-methylbutyrate or a salt thereof and the at least one acid are combined and undergo an acidification reaction to produce a second product stream comprising beta-hydroxy-beta-methylbutyrate in free acid form. While the specific example shown in FIG. 2 illustrates a second flow reactor (220) in fluid communication with the flow reactor (210), the second flow reactor (220) is optional, as the at least one acid may be combined with the product stream in the flow reactor (210) at a predetermined downstream location.

In the continuous process reactions disclosed herein, in those embodiments where the product stream and the at least one acid are combined and undergo the acidification reaction to produce the second product stream, the second product stream may be further processed. For example, in certain embodiments, a separation process is used to isolate the beta-hydroxy-beta-methylbutyrate in free acid form from the second product stream. To accomplish this isolation, certain embodiments of the third embodiment of the disclosed system further includes a continuous extractor in fluid communication with the flow reactor and a source of at least one organic solvent. As seen in FIG. 2, the second product stream is combined with at least one organic solvent (here ethyl acetate) in the continuous extractor to create an organic solvent phase. The at least one organic solvent is chosen such that the beta-hydroxy-beta-methylbutyrate in free acid form is preferentially soluble in the at least one organic solvent as compared to the second product stream. Thus, the organic solvent phase comprises beta-hydroxy-beta-methylbutyrate in free acid form and may be subjected to further processing, while a waste stream exits the continuous extractor for treatment and disposal or recycling.

As seen in FIG. 2, and in certain embodiments according to the third embodiment, the organic solvent phase comprising beta-hydroxy-beta-methylbutyrate in free acid form may be processed to recover the beta-hydroxy-beta-methylbutyrate in free acid form from the organic solvent phase. For example, in certain embodiments according to the third embodiment, the system comprises an evaporator in fluid communication with the continuous extractor such that the beta-hydroxy-beta-methylbutyrate in free acid form is recovered from the organic solvent phase. As briefly mentioned above, in certain embodiments the evaporator may be a thin film or wiped film evaporator. However, in alternative embodiments, the system may comprise a distillation column in fluid communication with the continuous extractor to recover beta-hydroxy-beta-methylbutyrate in free acid form from the organic solvent phase.

Referring again to FIG. 2, in those embodiments where the beta-hydroxy-beta-methylbutyrate in free acid form is recovered from the organic solvent phase, the beta-hydroxy-beta-methylbutyrate in free acid form may be subjected to further processing steps, such as a purification step. Thus, in certain embodiments of the system according to the third embodiment, the system further comprises a crystallizer in fluid communication with the evaporator, a source of at least one separation solvent, and at least one source of calcium cations. When the beta-hydroxy-beta-methylbutyrate in free acid form, the at least one recrystallization solvent, and the at least one source of calcium cations are combined in the crystallizer, a third product stream is produced comprising crystallized calcium beta-hydroxy-beta-methylbutyrate (or other salt-form of the beta-hydroxy-beta-methylbutyrate). As mentioned above, in certain embodiments according to the third embodiment of the system, the crystallizer comprises a continuous oscillatory baffled crystallizer. However, other types of crystallizers and crystallization systems may be utilized so long as they are capable of producing a third product stream comprising crystallized calcium beta-hydroxy-beta-methylbutyrate (or other salt-form of the beta-hydroxy-beta-methylbutyrate).

With continued reference to FIG. 2, in certain embodiments according to the third embodiment, after crystallized calcium beta-hydroxy-beta-methylbutyrate (or other salt-form of the beta-hydroxy-beta-methylbutyrate) is produced in the third product stream, the third product stream may be further processed to recover the crystallized calcium beta-hydroxy-beta-methylbutyrate. To accomplish this separation, certain embodiments of the system according to the third embodiment further comprise a continuous centrifugator in fluid communication with the crystallizer. The continuous centrifugator separates the crystallized calcium beta-hydroxy-beta-methylbutyrate (or other salt-form of the beta-hydroxy-beta-methylbutyrate) from the remaining components of the third product stream, which constitute the mother liquor. As described above, the mother liquor may be further processed to recover any residual calcium beta-hydroxy-beta-methylbutyrate (or other salt-form of the beta-hydroxy-beta-methylbutyrate). In addition, in certain other embodiments, the system may comprise a filtration apparatus or a decantation apparatus for recovering the crystallized calcium beta-hydroxy-beta-methylbutyrate (or other salt-form of the beta-hydroxy-beta-methylbutyrate).

Optionally, the recovered crystallized calcium beta-hydroxy-beta-methylbutyrate (or other salt-form of the beta-hydroxy-beta-methylbutyrate) may undergo a drying process to remove residual solvent. Thus, in certain embodiments of the system according to the third embodiment, the system comprises a continuous dryer in fluid communication with the continuous centrifugator, as shown in FIG. 2. The continuous dryer operates to remove residual solvent the recovered crystallized calcium beta-hydroxy-beta-methylbutyrate (or other salt-form of the beta-hydroxy-beta-methylbutyrate) to provide an even purer form of crystallized calcium beta-hydroxy-beta-methylbutyrate (or other salt-form of the beta-hydroxy-beta-methylbutyrate). However, as briefly mentioned above, it may not be possible to completely remove all residual solvent, thus the crystallized calcium beta-hydroxy-beta-methylbutyrate (or other salt-form of the beta-hydroxy-beta-methylbutyrate) may still contain some amount of residual solvent.

Although only the sodium salt and the calcium salt of beta-hydroxy-beta-methylbutyrate are explicitly discussed herein, the presently disclosed continuous processes and systems may be utilized to produce other salt forms of beta-hydroxy-beta-methylbutyrate, including alkali metal salts or alkaline earth metal salts or both. For example, the presently disclosed continuous processes and systems may be used to produce a calcium salt, a sodium salt, a potassium salt, a magnesium salt, a chromium salt, or combinations thereof.

The presently disclosed continuous processes and systems for manufacturing beta-hydroxy-beta-methylbutyrate or a salt thereof will be better understood by reference to the following examples, which are intended as an illustration of and not a limitation upon the scope of the inventive concept.

### EXAMPLES

The Examples provided below illustrate a comparison between different batch systems and the presently disclosed continuous processes for manufacturing beta-hydroxy-beta-methylbutyrate or a salt thereof. Examples 1, 2 and 3 are comparative examples.

### Example 1

Conventional batch mode preparation of beta-hydroxy-beta-methylbutyrate (HMB) from diacetone alcohol (DIA), as disclosed in U.S. Pat. No. 6,090,918, was reported to provide an average yield of 0.26 pounds of HMB per pound of DIA (i.e., 25.6% yield), with the most efficient batch achieving a yield of 0.325 pounds of HMB per pound of DIA (i.e., 32.0% yield). The reaction was typically run in a reactor no greater than 200 gallons, with an average charge of 156 gallons of sodium hypochlorite and about 95 pounds of DIA, to produce about 25 pounds of HMB per batch (i.e., 25.9% yield).

### Example 2

Batch mode preparation of beta-hydroxy-beta-methylbutyrate (HMB) from diacetone alcohol (DIA) employing the procedure and apparatus as disclosed in U.S. Patent No. 6,090,978, which is fully incorporated herein by reference, was reported to provide an average yield of 0.44 pounds of HMB per pound of DIA (i.e., 43.3% yield). The highest batch yield with the procedure of the '978 patent was reported to be 0.50 pounds of HMB per pound of DIA (i.e., 49.2% yield). The oxidation of DIA was conducted at a reported temperature of 3° C.-10° C. for a period of 30 minutes.

### Example 3

Batch mode preparation of beta-hydroxy-beta-methylbutyrate (HMB) was conducted in the laboratory to determine the batch mode yield at room temperature and reduced temperature, as well as under bleach rich and bleach lean conditions. As used herein, the term “bleach rich,” refers to a HMB synthetic procedure wherein diacetone alcohol (DIA) is added through controlled addition to a solution of oxidant, preferably sodium hypochlorite (NaClO). As used herein, the term “bleach lean,” refers to a HMB synthetic procedure wherein oxidant, preferably sodium hypochlorite, is added through controlled addition to DIA. In general, reactions were conducted with a bleach to DIA equivalence ratio ranging from about 3:1 to about 4:1. Reaction yields were determined via HPLC analysis, and more specifically, according to equation (1) wherein the concentration (moles/kg) of HMB in the reaction mixture (determined by HPLC) was multiplied by the weight of the reaction mixture (weight of DIA+weight of NaClO solution) and divided by moles of DIA charged in the experiment.

$$HMB yield = \frac{[ HMB ] in reaction mixture * Weight of reaction mixture}{Moles of DIA charged} \qquad ( 1 )$$

Under batch mode, room temperature, bleach rich operating conditions, 3 milliliters (ml) of DIA was added through controlled addition to 50 ml of 11.9% aqueous sodium hypochlorite solution, providing a 48%-50% yield of HMB as measured via HPLC analysis. Batch mode, room temperature, bleach rich operating conditions were found to generally provide a 48%-50% yield of HMB in about 12-20 minutes.

Under batch mode, room temperature, bleach lean operating conditions, 50 ml of 11.9% aqueous sodium hypochlorite solution was added through controlled addition to 3 ml of DIA, providing a 10%-12% yield of HMB as measured via HPLC analysis. Room temperature, bleach lean operating conditions were found to generally provide 10%-12% yield of HMB in about 12-20 minutes.

Under batch mode, reduced temperature (3° C.), bleach rich operating conditions, 3 ml of DIA was added through controlled addition to 50 ml of 11.9% aqueous sodium hypochlorite solution, providing a 60%-67% yield of HMB as measured via HPLC analysis. Batch mode, reduced temperature, bleach rich operating conditions were found to generally provide a 60%-67% yield of HMB in about 12-20 minutes.

Under batch mode, reduced temperature (3° C.), bleach lean operating conditions, 50 ml of 11.9% aqueous sodium hypochlorite solution was added through controlled addition to 3 ml of DIA, providing a 16%-24% yield of HMB as measured via HPLC analysis. Batch mode, reduced temperature, bleach lean operating conditions were found to generally provide a 16%-24% yield of HMB in about 12-20 minutes.

The batch mode process production results confirm the exothermic nature of the oxidation reaction of DIA, and that failure to control the temperature contributes to thermal degradation of HMB. The results further indicate that at high pH, as is present in the bleach lean conditions, DIA decomposition to acetone contributes to a low HMB yield as DIA reactant is consumed by a side reaction with sodium hydroxide byproduct produced from the oxidation. As will be discussed further below, the batch mode process also requires longer cycle times as compared to continuous process conditions because slow addition of reactants is necessary in the batch mode to maintain the desired reaction temperature and prevent thermal degradation of HMB product, DIA decomposition to acetone, or both.

### Example 4

Beta-hydroxy-beta-methylbutyrate (HMB) was prepared by a continuous process according to the present disclosure. In particular, the sodium salt of HMB (NaHMB) was prepared by a continuous flow process on a laboratory scale setup consistent with FIG. 1, using a tubular flow reactor purchased from Koflo Corporation, 309 Cary Point Drive, Cary, Ill. 60013. The reaction temperature and residence time were varied to evaluate HMB yield as a function of residence time and temperature. In general, reactions were conducted with a sodium hypochlorite (NaClO) to diacetone alcohol (DIA) equivalence ratio ranging from about 3:1 to about 4:1. The sodium hypochlorite used was an aqueous solution of 11.9% (by weight) sodium hypochlorite. The diacetone alcohol utilized was neat. Reaction yields were determined via HPLC analysis, and more specifically, according to equation (2) wherein the concentration (moles/kg) of HMB in the reaction mixture (determined by HPLC) was multiplied by the reaction flow rate (kg/hr) (determined by DIA flow rate+NaClO flow rate) and total reaction collection time (hr), and then divided by moles of DIA, which was determined by multiplying DIA flow rate (moles/hr) by total reaction collection time (hr).

$$HMB yield = \frac{\begin{matrix}[ HMB ] in rxn mixture * Rxn flow rate * \\ Total rxn collection time\end{matrix}}{DIA flow rate * Total rxn collection time}$$

Flow process production of HMB at room temperature (˜20° C.) and a residence time of 6.4 minutes generally provided an HMB yield of 46%-47%. Flow process production of HMB at room temperature (˜20° C.) and a residence time of 12.8 minutes generally provided an HMB yield of 46%-47%. Flow process production of HMB at reduced temperature (˜3° C.) and a residence time of 3.2 minutes generally provided an HMB yield of about 52%. Flow process production of HMB at reduced temperature (˜3° C.) and a residence time of 6.4 minutes generally provided an HMB yield of about 58%-76%. Flow process production of HMB at reduced temperature (3° C.) and a residence time of 12.8 minutes provided an HMB yield of 64%-78%.

The flow process production of HMB results indicate that the smaller thermal mass leads to better reaction control as compared to batch mode, which in turn leads to a higher yield of HMB. Shorter residence times, as compared to batch mode, also contributes to higher HMB yield because less NaHMB degradation or diacetone alcohol decomposition occurs. The flow process also has additional advantages of better thermal efficiency, lower energy consumption, and flexibility of scale-up as compared to the known batch mode processes. For example, the continuous process of the present disclosure may be easily scaled-up or down via adjusting the operating time of the process, or by adding or subtracting flow reactors.

Table 2, shown below, summarizes the results from Examples 1-4. The results indicate that the continuous processes of the present disclosure provide the aforementioned advantages over the known batch processes.

TABLE 2

| | | Result | | | | |
|---|---|---|---|---|---|---|
| Exam- | Process | Temper- | | | Bleach rich/ | |
| ple | Type | ature | Residence Time | | Bleach lean | Yield |
| 1 | Batch | ND | ND | | BR | 25-32% |
| 2 | Batch | LT | 30 | minutes | BR | 43-49% |
| 3 | Batch | RT | 12-20 | min | BR | 48-50% |
| | | RT | 12-20 | min | BL | 10-12% |
| | | LT | 12-20 | min | BR | 60-67% |
| | | LT | 12-20 | min | BL | 16-24% |
| 4 | Contin- | RT | 6.4 | min | N/A | 46-47% |
| | uous | RT | 12.8 | min | N/A | 46-47% |
| | Flow | LT | 3.2 | min | N/A | 52% |
| | | LT | 6.4 | min | N/A | 58-76% |
| | | LT | 12.8 | min | N/A | 61-78% |

BR = Bleach Rich; BL = Bleach Lean; RT = room temperature (~20° C.); LT = low temperature (~3° C.); Min = Minutes; ND = not determined

To the extent that the term “includes” or “including” is used in the specification or the claims, it is intended to be inclusive in a manner similar to the term “comprising” as that term is interpreted when employed as a transitional word in a claim. Furthermore, to the extent that the term “or” is employed (e.g., A or B) it is intended to mean “A or B or both.” When the applicants intend to indicate “only A or B but not both” then the term “only A or B but not both” will be employed. Thus, use of the term “or” herein is the inclusive, and not the exclusive use. See Bryan A. Garner, A Dictionary of Modern Legal Usage 624 (2d. Ed. 1995). Also, to the extent that the terms “in” or “into” are used in the specification or the claims, it is intended to additionally mean “on” or “onto.” Furthermore, to the extent the term “connect” is used in the specification or claims, it is intended to mean not only “directly connected to,” but also “indirectly connected to” such as connected through another component or components.

While the present application has been illustrated by the description of embodiments thereof, and while the embodiments have been described in considerable detail, it is not the intention of the applicants to restrict or in any way limit the scope of the appended claims to such detail. Additional advantages and modifications will readily appear to those skilled in the art. Therefore, the application, in its broader aspects, is not limited to the specific details, the representative compositions and processes, and illustrative examples shown and described. Accordingly, departures may be made from such details without departing from the spirit or scope of the applicant's general inventive concept.

What is claimed is:

1. A continuous process for manufacturing beta-hydroxy-beta-methylbutyrate or a salt thereof, comprising:
(A) providing at least one oxidant;
(B) providing diacetone alcohol, wherein an equivalence ratio of the at least one oxidant to the diacetone alcohol is within a range of 3:1 to 4:1; and
(C) combining the at least one oxidant with the diacetone alcohol in a flow reactor to form a product stream, wherein the temperature of the product stream is within a range of −10° C. to 40° C.

2. The continuous process according to claim 1, wherein the temperature of the product stream is within a range of −10° C. to 0° C.

3. The continuous process according to claim 1, wherein the at least one oxidant is at a temperature of −20° C. to 20° C. prior to or upon being combined with the diacetone alcohol, and the diacetone alcohol is at a temperature of −20° C. to 20° C. prior to or upon being combined with the at least one oxidant.

4. The continuous process according to claim 3, wherein the at least one oxidant is selected from the group consisting of sodium hypochlorite, calcium hypochlorite, calcium hypobromite, calcium hypoiodite, sodium hypobromite, sodium hypoiodite, and combinations thereof.

5. The continuous process according to claim 1, wherein the at least one oxidant and the diacetone alcohol remain in the flow reactor for 3 minutes to 20 minutes.

6. The continuous process according to claim 1, further comprising collecting the product stream, wherein the product stream comprises a salt of beta-hydroxy-beta-methylbutyrate.

7. The continuous process according to claim 1, further comprising combining the product stream with at least one acid to form a second product stream having a temperature of −5° C. to 5° C. and a pH of less than 5, wherein the second product stream comprises beta-hydroxy-beta-methylbutyrate in free acid form.

8. The continuous process according to claim 7, wherein the at least one acid is selected from the group consisting of hydrogen chloride gas, hydrochloric acid, hydrobromic acid, hydroiodic acid, sulfuric acid, bromic acid, and combinations thereof.

9. The continuous process according to claim 1, wherein the flow reactor comprises a tubular reactor having one or more static mixing elements.

10. A continuous process for manufacturing calcium beta-hydroxy-beta-methylbutyrate, comprising:
(A) combining at least one oxidant with diacetone alcohol in a flow reactor to form a product stream having a temperature of −10° C. to 40° C., wherein an equivalence ratio of the at least one oxidant to the diacetone alcohol is within a range of 3:1 to 4:1, and the product stream comprises a salt of beta-hydroxy-beta-methylbutyrate;
(B) combining the product stream with at least one acid to form a second product stream having a temperature of −5° C. to 5° C., wherein the second product stream comprises beta-hydroxy-beta-methylbutyrate in free acid form;
(C) combining the second product stream with at least one organic solvent to create an organic solvent phase, wherein the beta-hydroxy-beta-methylbutyrate in free acid form is preferentially soluble in the organic solvent phase;
(D) removing a majority of the at least one organic solvent from the organic solvent phase to produce a concentrated organic solvent-product phase comprising beta-hydroxy-beta-methylbutyrate in free acid form;
(E) mixing the concentrated organic solvent-product phase comprising beta-hydroxy-beta-methylbutyrate in free acid form with at least one source of calcium cations to form a third product stream comprising calcium beta-hydroxy-beta-methylbutyrate, wherein the third product stream has a pH of at least 6; and
(F) recovering the calcium beta-hydroxy-beta-methylbutyrate from the third product stream.

11. The continuous process according to claim 10, wherein the temperature of the product stream is within a range of −10° C. to 0° C.

12. The continuous process according to claim 10, wherein the at least one oxidant is at a temperature of −20° C. to 20° C. prior to or upon being combined with the diacetone alcohol, and the diacetone alcohol is at a temperature of −20° C. to 20° C. prior to or upon being combined with the at least one oxidant.

13. The continuous process according to claim 12, wherein the at least one oxidant is selected from the group consisting of sodium hypochlorite, calcium hypochlorite, calcium hypobromite, calcium hypoiodite, sodium hypobromite, sodium hypoiodite, and combinations thereof.

14. The continuous process according to claim 10, wherein the at least one acid is selected from the group consisting of hydrogen chloride gas, hydrochloric acid, hydrobromic acid, hydroiodic acid, sulfuric acid, bromic acid, and combinations thereof.

15. The continuous process according to claim 10, wherein the at least one organic solvent is selected from the group consisting of ethyl acetate, diethyl ether, and combinations thereof.

16. The continuous process according to claim 10, wherein the at least one source of calcium cations is selected from the group consisting of calcium hydroxide, calcium oxide, calcium carbonate, calcium acetate, and combinations thereof.

17. The continuous process according to claim 10, further including providing a recrystallization solvent for mixing with the concentrated organic solvent-product phase and the at least one source of calcium cations, wherein the recrystallization solvent is selected from the group consisting of ethanol, ethyl acetate, acetone, water, and combinations thereof.

18. A system for manufacturing beta-hydroxy-beta-methylbutyrate or a salt thereof, comprising:
(A) a first pump in fluid communication with (i) a source of at least one oxidant, and (ii) a first heat exchanger;
(B) a second pump in fluid communication with (i) a source of diacetone alcohol, and (ii) a second heat exchanger; and
(C) a flow reactor in fluid communication with the first heat exchanger and the second heat exchanger;
whereby the at least one oxidant and the diacetone alcohol undergo an oxidation reaction in the flow reactor to produce a product stream comprising beta-hydroxy-beta-methylbutyrate or a salt thereof.

19. The system of claim 18, further comprising:
(A) a third pump in fluid communication with a source of at least one acid and the flow reactor, wherein the product stream comprising beta-hydroxy-beta-methylbutyrate or a salt thereof and the at least one acid undergo an acidification reaction to produce a second product stream comprising beta-hydroxy-beta-methylbutyrate in free acid form;
(B) a continuous extractor in fluid communication with (i) the flow reactor, and (ii) a source of at least one organic solvent, wherein the second product stream is combined with at least one organic solvent in the continuous extractor to create an organic solvent phase, wherein the beta-hydroxy-beta-methylbutyrate in free acid form is preferentially soluble in the at least one organic solvent;
(C) an evaporator in fluid communication with the continuous extractor, wherein beta-hydroxy-beta-methylbutyrate in free acid form is recovered from the organic solvent phase;
(D) a crystallizer in fluid communication with (i) the evaporator, (ii) a source of at least one separation solvent, and (iii) at least one source of calcium cations, wherein the beta-hydroxy-beta-methylbutyrate in free acid form, the at least one recrystallization solvent, and the at least one source of calcium cations are combined to produce a third product stream comprising crystallized calcium beta-hydroxy-beta-methylbutyrate;
(E) a continuous centrifugator in fluid communication with the crystallizer, wherein the crystallized calcium beta-hydroxy-beta-methylbutyrate is recovered from the third product stream; and
(F) a continuous dryer in fluid communication with the continuous centrifugator, wherein residual solvent is removed from the recovered crystallized calcium beta-hydroxy-beta-methylbutyrate.
